(19) United States
(12) Patent Application Publication
(10) Pub. No.: US 2005/0148661 A1
(43) Pub. Date: Jul. 7, 2005

(54) Protection against the neurotoxicity of oxaliplatin through the administration of calcium and magnesium

(75) Inventors: Laurence Gamelin, Bouchemaine (FR); Erick Gamelin, Bouchemaine (FR); Michele Boisdron-Celle, Saint-Leger-Des-Bois (FR); Alain Morel, Juigne Sur Loire (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Centre Regional de Lutte Contre le Cancer, Angers (FR)

(21) Appl. No.: 10/501,318

(22) PCT Filed: Jan. 14, 2003
(86) PCT No.: PCT/FR03/00098

(30) Foreign Application Priority Data
Jan. 14, 2002 (FR) 02/00390

(51) Int. Cl. A61K 31/28; A61K 31/19

(52) U.S. Cl. 514/492; 514/574; 514/557

(57) ABSTRACT

The invention relates to products comprising calcium, injectable magnesium and an injectable product which releases oxalate during the metabolism thereof as a combination that can be used for simultaneous, sequential or separate administration in anticancer and antiviral therapies.

### CROSS REFERENCE TO RELATED APPLICATIONS

This application is a national stage filing under 35 U.S.C. §371 of PCT/FR03/00098 filed Jan. 14, 2003, which claims priority from French patent application no. 02/00390, filed Jan. 14, 2002.

### BACKGROUND OF THE INVENTION

Platinum derivatives have revolutionized the treatment of certain types of cancer.

A novel organic derivative of platinum which was developed fairly recently, 1,2-diamine cyclohexane (trans-1)oxalatoplatinum, or oxaliplatin, has proved very active in digestive tumors (Misset et al., La Lettre du Cancérologue (1996), 5, 20-22). It received marketing authorization as first-line therapy for metastatic colorectal cancers. It is under study in an adjuvant situation and in other tumors: pancreas, stomach, lungs and ovaries. It is therefore very frequently used.

Free of renal toxicity, it is responsible for a peripheral neurotoxicity, which is its limiting toxicity. This is a peripheral neuropathy, very different from that of cisplatin, the latter being chronic, cumulative, setting in gradually and most often irreversibly (Mollman, Cisplatin Neurotoxicity, (1990), 322, 126-127). The neurotoxicity of oxaliplatin manifests itself in acute and/or chronic form, sometimes highly incapacitating. The acute attack is quite novel and is characterized by its sudden onset, during intravenous infusion or immediately afterwards. It manifests itself by paresthesia of the extremities and/or perioral paresthesia, dysesthesia exacerbated by exposure to cold, cramps which are sometimes difficult to reduce. Acute, early and transient paresthesia caused by cold affects the fingers, the hands, the tongue and the lips. It gives a picture of parapanesis of the lower limbs. Peripheral neuropathy is reported at least once by 85 to 95% of patients during their treatment. It usually appears during infusion and can last for several days. Its duration increases with the number of treatment cycles. It is dose-dependent and cumulative since the frequency of the grade 3 neuropathy is 15-20% after a cumulative dose of 750-850 mg/m² (Berthault-Cvitkovic et al., J. Clin. Oncol. (1996), 14, 2950-8). This toxicity may go as far as a pseudolaryngospasm. This attack, which is sometimes highly incapacitating and agonizing, is transient and regresses within a few hours or days.

Neuropathy of chronic expression for its part resembles, in its manifestations and its progression, that caused by cisplatin but it occurs much later, after several administrations of oxaliplatin (Machover et al., Ann. Oncol. (1996), 7, 95-8). It follows acute toxicity, can last for several months, or can even not regress, thereby affecting the quality of life of patients who experience serious difficulties writing, buttoning up, lacing their shoes, wearing smart shoes, and walking. Histological studies have not shown neuronal impairment which is as evident as with cisplatin, myelin is rarely affected, Wallerian degeneration exceptional (Raymond), whereas with cisplatin, biopsies have shown infiltration of the nerve tissue by platinum, with segmental disappearance of the myelin sheath and Wallerian degeneration (Mollman, Cisplatin Neurotoxicity, N. Engl. J. Med. (1990), 322, 126-127).

These problems of toxicity limit the use of oxaliplatin and can even lead to the treatment being stopped. Accordingly, it is essential to improve the tolerance of oxaliplatin which has proved essential in the treatment of certain cancers.

The first stage of the metabolism of oxaliplatin consists in the release of oxalate which is replaced by 2 chlorine ions, thus resulting in the formation of dichlorodachplatin (dichlorodiaminocyclohexane-platinum). This reaction is carried out at 30% in the plasma medium and at 70% in the intracellular medium. Oxalate is known in toxicology for its calcium and magnesium chelating capacity (Hagler et al., Oxalate Metabolism, (1973), 26, 1073-1079 and L'Epée et al, Intoxication aiguë mortelle par l'oxalate de potassium. Med. Led. Dom. Corp. (1971), 4, 178-181) and an acute oxalate poisoning results in paresthesia, myoclonia, which can go as far as convulsions in the case of massive poisoning. Hypocalcemia and metabolic acidosis has been reported and renal toxicity is possible, due to tubular precipitation of oxalate crystals.

Accordingly, the authors have put forth the hypothesis of the role of the chronic inhibition of sodium channels in the genesis of the neurotoxicity of oxaliplatin. Indeed, ion channels play a major role in neuronal cellular homeostasis and the long-term inhibition of sodium channels can hamper ion movements and modify the concentrations of ions and intracellular constituents. Now, the latter are essential for vital processes such as the release of neurotransmitters, elongation of the axon growth cone, and gene expression. Prolonged inhibition of the neurosecretory function and of neuritic development can have long-term deleterious consequences (Rizzo et al, Mechanisms of paraesthesiae, dysesthesiae and hyperesthesiae: Role of Na Channel heterogeneity).

An electrophysiological study was carried out in vitro by:
a) current and voltage clamp techniques on axon isolated from cockroaches with the double oil separation technique,
b) patch clamp techniques in whole cell configuration on DUM neurons (dorsal unpair median neuron) from cockroaches (DEA Laurence Gamelin, Etude de la neurotoxicité d'un dérivé du platine, l'oxaliplatine, par une approche électrophysiologique, Paris VII (1999)).

On axon isolated from cockroaches, oxaliplatin, applied externally, caused no significant modification in the sodium or potassium ion currents, unlike tetrodotoxin, a reference substance for studying the inhibition of sodium channels as a whole. Using the patch clamp technique in whole cell configuration, a reduction in the amplitude of the action potential over time was obtained in the presence of oxaliplatin, in current clamp, as well as a 50% reduction in the sodium current over 10 minutes in voltage clamp, reaching a plateau of 60% sodium current reduction. The inventors have thus been able to show that oxaliplatin substantially reduces the incoming current of Na, and therefore reduces the action potential of the neuron. The concentrations necessary for the inhibition of the sodium channels are of the order of magnitude of the concentrations obtained in the first few hours of infusion of oxaliplatin in human clinical medicine.

The effect of oxaliplatin on the calcium channels was also examined, but no significant effect was found. It exerts some inhibitory action on the sodium channels by the intracellular route but its action differs from tetrodotoxine by a less effective IC₅₀ (with a concentration ratio of 10⁻⁴) since a plateau occurring at 60% inhibition is obtained. The results show that it acts on some calcium-dependent ion channels. Indeed, BAPTA, a calcium chelator, gave an identical sodium channel inhibition curve. The inventors have been able to show that oxaliplatin acts via oxalate, blocking some calcium-dependent sodium channels involved in the transmission of the nerve impulse (FIG. 2). This blocking of the sodium channels causes abnormalities in the action potential of the neuron, that is to say neuronal depolarization disorders and potentially disturbances in the transmission of the impulse.

Other platinum salts were tested: cisplatin, carboplatin and Dach platin, a metabolite of oxaliplatin, exert no effect.

The inventors therefore set themselves the aim of using calcium and magnesium for increasing tolerance to oxaliplatin.

### SUMMARY OF THE INVENTION

The subject of the present invention is a pharmaceutical composition based on calcium and magnesium which reduces the neurotoxicity of the active ingredients which release oxalate during their metabolism in the body, in particular that of oxaliplatin.

Thus, the subject of the present invention is products comprising calcium, injectable magnesium and an active ingredient which releases oxalate during its metabolism in the body, as a combination useful for simultaneous, sequential or separate administration in anticancer or antiviral therapy.

In an advantageous embodiment of the invention, part of the calcium is in injectable form and the other part is in oral form.

In another advantageous embodiment of the invention, the active ingredient which releases oxalate is oxaliplatin.

### BRIEF DESCRIPTION OF THE DRAWINGS

FIG. 1 illustrates the metabolism of oxaliplatin. Oxaliplatin penetrates at 70% unchanged into the cell and is metabolized to diaminecyclohexaneplatinum (Dach platin) and oxalate.

FIG. 2 illustrates the mechanism of action of oxaliplatin on the sodium channels. Oxaliplatin penetrates into the cell and is metabolized, giving Dach platin, a cytotoxic active metabolite and oxalate.

FIG. 3 illustrates the duration of the treatment according to the prevention by calcium and magnesium and the frequency and intensity of distal neuropathy at the end of the treatment according to the infusion of calcium and magnesium. Abbreviations: Tox: toxic effects; PD: progressive disease; SD: stable disease; OR: objective response; neuro: neuropathy; thrombo: thrombopenia; and neutro: neutropenia.

### DETAILED DESCRIPTION OF THE INVENTION

For the purposes of the present invention, the expression calcium and magnesium is understood to mean the salified forms of these ions, in particular calcium gluconate, calcium chloride, bromogalactogluconate, calcium gluconolactate, calcium carbonate, magnesium sulfate and magnesium pidolate.

In an advantageous embodiment of the invention, the products are characterized in that the concentrations of injectable calcium are between 8 and 20 mg/ml and the concentrations of injectable magnesium are between 10 and 20 mg/ml, preferably 15 mg/ml (these concentrations are expressed as calcium ions).

The concentrations of calcium and magnesium salts are chosen so as to allow intravenous administration of 2 to 3 g/day of said salts during the administration of oxaliplatin. The calcium concentrations are chosen so as to allow administration of 1 to 2 g/day per os during the eight days which follow.

The subject of the invention is also the use of calcium and magnesium for the preparation of a combination product intended to prevent or treat neurotoxicity caused by the administration of a product which releases oxalate during its metabolism.

For the purposes of the present invention, the agents which release oxalate may be, apart from oxaliplatin, indinavir, ritonavir and their analogues, inhibitors of the human immunodeficiency virus (HIV) (Stoller M L, Curv. Opin. Urol. (2000), 10, 557-561).

The expression injectable form is understood to mean, for the purposes of the present invention, any liquid form capable of transporting the composition into the human body of a patient, such as for example isotonic solutions.

The expression oral form is understood to mean any form suitable for oral administration, in particular tablets, capsules and solutions.

The calcium and the magnesium may be used at any effective concentration which makes it possible to increase oxaliplatin tolerance; infusion in serum at 5% glucose containing 1 g of calcium gluconate and 1 g of magnesium sulfate before and after oxaliplatin gives good results.

The administration of calcium by the oral route may also be carried out at any dose which makes it possible to obtain the desired effect, in particular at a dose of 1 g/day by the oral route for the 8 days which follow the treatment with oxaliplatin.

In patients treated with oxaliplatin for digestive tumors, 1 gram of calcium gluconate and 1 gram of magnesium sulfate were administered by rapid infusion in the serum at 5% glucose, before and after oxaliplatin. It has been possible to observe a real efficacy of these infusions, first curatively for acute neurotoxic manifestations, and then preventively. The infusion of calcium and magnesium caused the acute neuropathy to regress very rapidly and for a long time. Administered preventively, before and after infusion of oxaliplatin, it massively reduces the onset of manifestations of neurotoxicity.

In patients treated with oxaliplatin, it has been possible to show significant concentrations of oxalate in plasma, normally undetectable, and substantial urinary elimination of oxalate within 5 hours following the start of infusion of oxaliplatin, accompanied by a rise in calciuria, kaliuria and magnesuria, but without any variation in the plasma concentrations of calcium (total, ionized) and of magnesium (total and overall). A fine intracellular disequilibrium can therefore be assumed in calcium homeostasis.

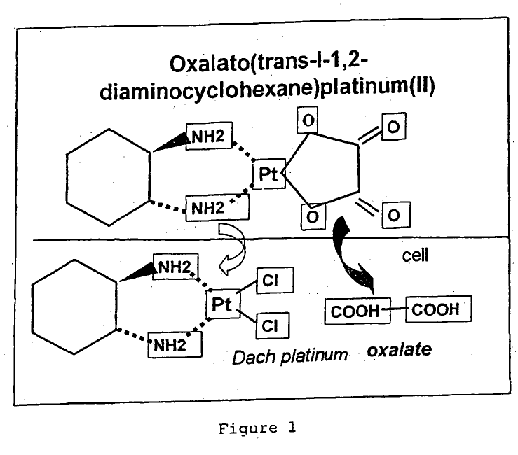

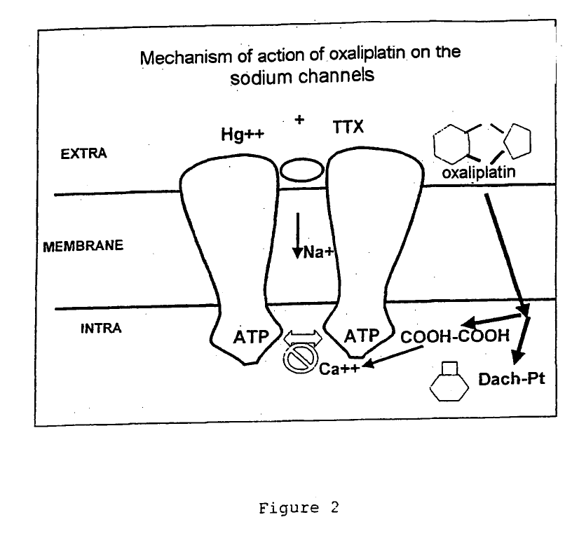

FIGS. 1 to 3 and the examples of clinical cases illustrate the invention.

### EXAMPLES

Prevention of the Neurotoxicity of Oxaliplatin by Calcium and Magnesium

1. Methodology

103 treatments comprising oxaliplatin were carried out according to the following scheme:
FOLFOX 4: oxaliplatin 85 mg/m²/15 d+fluorouracil and folinic acid (FUFOL),
FOLFOX 6: oxaliplatin 100 mg/m²/15 d+FUFOL,

FUFOL-LOHP: oxaliplatin 130 mg/m²/21 d+FUFOL, each treatment representing respectively 20%, 22% and 58% of the patients treated.

The tolerance of the treatment was evaluated and the toxic manifestations were quantified according to the NCI-CTC neurotoxicity scale and the specific scale for oxaliplatin which are illustrated in Table 1 below.

TABLE 1

Neurotoxicity scale

| | NCI-CTC | Specific scale |
|---|---|---|
| Grade 1 | Mild paresthesia, loss of | Paresthesia, dysesthesia |
| | R.O.T. | of short duration |
| Grade 2 | Moderate paresthesia, | Paresthesia, dysesthesia |
| | objective sensitive loss | persisting between |
| | | cycles |
| Grade 3 | Paresthesia with | Paresthesia, dysesthesia |
| | functional impairment, | with functional |
| | severe sensitive loss | impairment |

2. Results

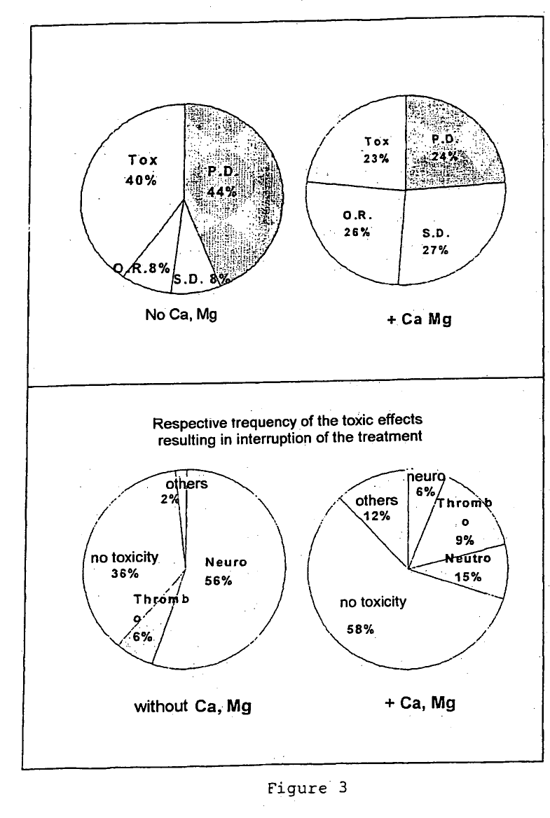

They are illustrated in the appended FIG. 3.

In the group of patients without calcium or magnesium, 40% of the discontinuations are due to toxic causes and 44% are linked to progression of the disease. In the group with calcium and magnesium, there is less toxicity and the reasons for therapeutic discontinuation are significantly fewer. A response or a stability of the disease is observed more often. The toxic causes for discontinuation are in the group without calcium and magnesium, mainly neuropathy (56%), while in the group with calcium and magnesium, the other conventional chemotherapy toxicities appear, neutropenia (15%), thrombopenia (9%). The administration of calcium and magnesium makes it possible to avoid toxic effects and to carry out the treatment at full doses while observing periods between the treatment. It modifies the oxaliplatin tolerance profile which becomes that of a well-tolerated chemotherapy molecule. This results in a better efficacy.

The infusion of calcium and magnesium makes it possible to increase the total duration of the treatments, when the latter are effective.

At the end of the chemotherapy, regardless of its duration, the frequency and intensity of the distal neuropathy are significantly lower in the case of infusion of calcium and magnesium (p<0.001).

During the treatment, at cycles 1, 3, 6 and at the end of the treatment, regardless of the cycle, the frequency and intensity of distal neuropathy are significantly less in the group of patients receiving calcium and magnesium. At cycle 6, 40% of the patients have no neurological manifestation against 12% in the absence of calcium and magnesium. Likewise, with calcium and magnesium, there is no grade 3, and therefore incapacitating chronic, toxicity. The number of patients without calcium or magnesium decreases gradually from cycle 1 to cycle 6: because a number of them then receive calcium and magnesium, because the others interrupt their treatment because of a neuropathy.

With the specific scale, the results are equivalent to the NCI-CTC scale. The specific oxaliplatin scale is based not only on the functional effect of the neuropathy but also on its duration. The infusion of calcium and magnesium significantly reduces (p<0.001) the intensity of the distal neuropathy, its duration and its functional effect.

This study shows that:
the infusion of calcium and magnesium makes it possible to significantly reduce the intensity and the duration of all the acute neurotoxic manifestations of oxaliplatin,
the infusion of calcium and magnesium also significantly reduces chronic toxicity, in terms of incidence and intensity,
oxaliplatin can be reintroduced after prolonged treatment at a total dose of up to 780 mg/m², even in the case of chronic persistent neuropathy, as long as it is combined with infusion of calcium and magnesium.

In some cases, it is found to be necessary to continue with the administration of Ca in order to reduce the risk of onset of neurological manifestations at a distance from the administration of oxaliplatin. In these cases, calcium, by the oral route, is effective and makes it possible to preserve the quality of life of the patients.

Clinical Case 1

A 58-year-old patient is treated with the combination 5-fluororuracil, folinic acid, oxaliplatin, 130 mg/m²/21 d.

During the first 2 cycles, she received a calcium and magnesium infusion before and after oxaliplatin. The neurological effects consist, at the 2nd cycle, of a grade 1 peripheral neuropathy on the specific scale.

At the 3rd cycle, the prevention by calcium and magnesium is forgotten. The patient then suddenly has at the end of the infusion grade 3 peripheral and perioral acute neurotoxicity (NCI-CTC and specific scale), pharyngolaryngeal manifestations, cramps in particular in the jaws and the hands.

During the next cycle, the prevention by calcium and magnesium is carried out and the treatment is very well tolerated.

Again, this is forgotten at cycle 5 and the very major acute neurological manifestations reappear. The 6th cycle with calcium and magnesium is very well tolerated.

Clinical Case 2

Another 60-year-old patient was treated for a metastatic colon cancer.

Her treatment consisted of the combination 5-FU folinic acid+oxaliplatin 130 mg/m²/21 days.

During cycle 1, very rapid appearance of distal and perioral grade 3 paresthesia, cramps in the jaws and the legs, cramp in the hands, accompanied by numbness, pseudolaryngospasm, dyspnea, asthenia and grade 3 diarrhea.

During cycle 2, the undesirable effects are identical, disappearing within a few minutes with an infusion of 1 g of calcium and magnesium i.v.: distal paresthesia occurs from grade 1 (NCI and specific scale).

At the third cycle, with calcium and magnesium as an infusion for preventive purposes, there is no neurotoxicity or diarrhea.

Clinical Case 3

Another 66-year-old patient was treated for a metastatic colon cancer.

He received as a first line of chemotherapy the combination 5-FU, folinic acid+oxaliplatin at the dose of 130 mg/m²/3 weeks, without infusion of calcium and magnesium.

During cycles 1 and 2, appearance of distal, perioral paresthesia of grade 2 NCI and of grade 1 on the specific scale.

At cycles C3, C4, C5, the neuropathy worsens and becomes grade 2 NCI and 2 on the specific scale.

At stage C6, the neuropathy continues to worsen and becomes grade 3 NCI and 3 on the specific scale: chronic neuropathy, cramps in the hands; common and very bothersome clinical effect: for writing, buttoning up, tying up his shoe laces. The very bothersome and incapacitating neuropathy lasts for 18 months.

Two years later, the patient observed a quite significant improvement, one of his neurological disorders, the metastatic tumor progresses again.

The same chemotherapy treatment is repeated, with oxaliplatin according to the same scheme at 130 mg/m²/3 weeks.

Six cycles of this protocol are carried out, with infusions of calcium and magnesium before and after infusion of oxaliplatin. No neuropathy is reported or observed.

1. A combination useful for simultaneous, sequential or separate administration in anticancer or antiviral therapy, comprising calcium, injectable magnesium and an active ingredient which releases oxalate during its metabolism.

2. The combination of claim 1, wherein part of the calcium is in injectable form and the other part is in oral form.

3. The combination claims 1 or, wherein said active ingredient is oxaliplatin.

4. The combination of claims 1, wherein the calcium is in gluconate, chloride, bromogalactogluconate, gluconolactate or carbonate form, and the magnesium is in sulfate or pidolate form.

5. The combination of claim 4, wherein part of the calcium is in injectable form and the other part is in oral form, and wherein said of injectable calcium is present in a concentration between 8 and 20 mg/ml, and wherein said injectable magnesium is present in a concentration between 10 and 20 mg/ml, concentrations being as calcium ions.

6. A method of preventing or treating neurotoxicity caused by the administration of a product that releases oxalate during its metabolism, comprising administering to a patient a combination of calcium and magnesium effective to prevent or treat the neurotoxicity.

7. The method of claim 6, wherein part of the calcium is administered to the patient in injectable form and the other part is administered in oral form.

8. The method of claim 6, where the product which releases oxalate is oxaliplatin.

9. The method of claim 7, wherein the calcium and magnesium are administered to the patient prior to, after, or both prior to and after administration of the product that releases oxalate during its metabolism.

10. The method of claim 7, wherein the calcium and magnesium are administered to the patient simultaneously with administration of the product that releases oxalate during its metabolism.

11. The method of claim 7, wherein said calcium and magnesium are administered separately.

12. The combination of claim 5, wherein the concentration of said injectable magnesium is 15 mg/ml.
